(19) United States
(12) Patent Application Publication
(10) Pub. No.: US 2025/0347674 A1
(43) Pub. Date: Nov. 13, 2025

(54) SYSTEMS AND METHODS FOR MEASURING THE PHYSICAL PROPERTIES OF LIQUIDS

(71) Applicant: Northrop Grumman Systems Corporation, Falls Church, VA (US)

(72) Inventor: Grant Camden Miars, San Diego, CA (US)

(21) Appl. No.: 18/660,315

(22) Filed: May 10, 2024

(51) Int. Cl. G01N 33/18; G01N 27/60; G01R 1/067

(52) U.S. Cl. CPC G01N 33/1886; G01N 27/60; G01N 33/1813; G01N 33/182; G01R 1/06738; G01R 1/06755

(57) ABSTRACT

Systems and methods for measuring the physical properties of liquids are described. A sensor system includes a measurement conductor having a defined geometry and configured to be placed in contact with a test liquid. The system includes control circuitry configured to bias the conductor to two or more voltages, determine a collected ion current to the conductor at the two or more voltages, determine at least one voltage-current characteristic based at least in part on the two or more voltages and the collected current to the conductor at the two or more voltages, determine at least one physical property of the liquid proximate to the conductor based at least in part on the at least one current-voltage characteristic, and generate at least one output indicative of the at least one physical property of the liquid proximate to the conductor.

### FIELD

The present disclosure relates generally to systems and methods for measuring the physical properties of water and other liquids.

### BACKGROUND

There is a significant need to test the physical properties of water and other liquids for a wide range of research and industrial applications including scientific, human safety, environmental, healthcare, chemical, manufacturing, and others. To meet these needs, many different types of instruments to measure the various physical properties of liquids have been developed. Typically, these instruments are capable of measuring a single physical property of a test liquid at a time. By way of example, sliver chloride reference electrode systems can use chemical reactions to roughly determine a local liquid potential. Such systems, however, require regular maintenance, are not physically robust, and are not capable of measuring other liquid properties. Salinometer systems can exploit chemistries and conductivities to determine salinity but are not capable of measuring voltage and temperature. While instruments capable of measuring multiple physical properties of liquids have been proposed, these systems typically have complex probes and are oftentimes fragile or bulky.

Accordingly, there is a need for improved systems and methods for measuring the physical properties of liquids such as water.

### BRIEF DESCRIPTION

Aspects and advantages of the disclosed technology will be set forth in part in the following description, or may be obvious from the description, or may be learned through practice of the disclosed embodiments.

One example aspect of the present disclosure is directed to a method of measuring physical properties of liquids. The method includes providing a conductor of a defined geometry in contact with a liquid, biasing the conductor to two or more voltages, determining a collected current to the conductor at the two or more voltages, determining at least one voltage-current characteristic based at least in part on the two or more voltages and the collected current to the conductor at the two or more voltages, determining at least one physical property of the liquid proximate to the conductor based at least in part on the at least one current-voltage characteristic, and generating at least one output indicative of the at least one physical property of the liquid proximate to the conductor.

Another example aspect of the present disclosure is directed to a sensor system to measure physical properties of liquids. The system includes a conductor having a defined geometry and control circuitry in electrical communication with the conductor. The control circuitry is configured to bias the conductor to two or more voltages, determine a collected current to the conductor at the two or more voltages, determine at least one current-voltage characteristic based at least in part on the two or more voltages and the collected current to the conductor at the two or more voltages, determine at least one physical property of the liquid proximate to the conductor based at least in part on the at least one current-voltage characteristic, and generate at least one output indicative of the at least one physical property of the liquid proximate to the conductor.

Yet another example aspect of the present disclosure is directed to one or more non-transitory computer-readable media that store instructions that, when executed by one or more processors, cause the one or more processors to perform operations. The operations include biasing a conductor to two or more voltages while in contact with a liquid, determining a collected ion current to the conductor at the two or more voltages, determining at least one current-voltage characteristic based at least in part on the two or more voltages and the collected current to the conductor at the two or more voltages, determining at least one physical property of the liquid proximate to the conductor based at least in part on the at least one current-voltage characteristic, and generating at least one output indicative of the at least one physical property of the liquid proximate to the conductor.

These and other features, aspects and advantages of the disclosed technology will become better understood with reference to the following description and appended claims. The accompanying drawings, which are incorporated in and constitute a part of this specification, illustrate embodiments of the disclosed technology and, together with the description, serve to explain the principles of the disclosed technology.

### BRIEF DESCRIPTION OF THE DRAWINGS

A full and enabling disclosure of embodiments, including the best mode thereof, directed to one of ordinary skill in the art, is set forth in the specification, which makes reference to the appended figures, in which:

FIG. 1A is a block diagram depicting an example sensor system configured to measure one or more physical properties of liquids in accordance with example embodiments of the present disclosure;

FIG. 1B is a block diagram depicting an example implementation of control circuitry for a sensor system to measure the physical properties of liquids according to example embodiments of the present disclosure;

FIG. 2A is a diagram depicting an example current-voltage curve of a liquid as determined by a sensor system according to an example embodiment of the present disclosure;

FIG. 2B is a diagram depicting an example I-V curve derivative based on a derivative of the I-V curve depicted in FIG. 2A;

FIG. 3 is a diagram depicting an example I-V curve of a test plasma measured by conductor biasing;

FIG. 4 is a block diagram of a test environment including a sensor system for measuring the physical properties of liquids in accordance with example embodiments of the present disclosure;

FIG. 5 is a diagram including a graphical depiction of example test results from a test including a sensor system for measuring the physical properties of liquids in accordance with example embodiments of the present disclosure;

FIG. 6 is a flowchart depicting an example method for measuring the physical properties of liquids in accordance with example embodiments of the present disclosure; and

FIG. 7 is a block diagram depicting an example of a computing system in accordance with embodiments of the present disclosure.

### DETAILED DESCRIPTION

Reference now will be made in detail to embodiments of the disclosed technology, one or more examples of which are illustrated in the drawings. Each example is provided by way of explanation of the disclosed embodiments, not limitation of the disclosed technology. In fact, it will be apparent to those skilled in the art that various modifications and variations can be made in the present disclosure without departing from the scope or spirit of the claims. For instance, features illustrated or described as part of one embodiment can be used with another embodiment to yield a still further embodiment. Thus, it is intended that the present disclosure covers such modifications and variations as come within the scope of the appended claims and their equivalents.

Generally, the present disclosure is directed to a sensor system configured to measure one or more physical properties of a liquid. More particularly, a sensor system is provided that includes at least one measurement electrode having a conductor with a defined geometry and that is configured to be placed into contact with a liquid. The system includes control circuitry configured to bias the conductor to multiple voltages and to measure an ion current collected by the conductor at the different voltages. The control circuitry is configured to determine and analyze a resulting current-voltage (I-V) characteristic such as an I-V curve based on the bias voltages and the resulting ion currents. The control circuitry can determine one or more properties of the liquid based on analyzing the resulting I-V curve. By way of example, the control circuitry can measure liquid properties including local voltage, ionic concentration, ionic composition, and/or temperature. The control circuitry can generate one or more outputs indicative of the measured liquid properties.

In accordance with example embodiments of the present disclosure, a sensor system is provided that includes a single probe or electrode having a conductor capable of determining commonly measured liquid properties. The electrode can include a simple and mechanically robust conductor having a simple, defined geometry. By way of example, the conductor can have a spherical, cylindrical, or planar geometry. The conductor can be electrically coupled to control circuitry that is configured to bias the conductor and determine precise collected current measurements that result from the biasing. For example, the control circuitry can apply a voltage sweep including multiple voltages and determine precise measurements of the total collected ion current at the conductor at each voltage step of the voltage sweep. The control circuitry can determine a resulting I-V curve based on the precise measurements and determine one or more physical properties of the liquid based on the resulting I-V curve. For example, the control circuitry can include or otherwise access one or more models of ion flow corresponding to the test liquid and conductor as part of its physical property determination analysis. The control circuitry can use the resulting I-V curve to measure one or more physical properties such as liquid electric potential, ion concentration (e.g., water salinity), ion composition, and liquid temperature.

According to an example implementation of the disclosed technology, a sensor system is provided that includes an electrically conducting object configured to passively collect charged particles (e.g., positively-charged ions and negatively-charged ions) to its surface from its environment. The collected current is capable of prediction based on the properties of the medium it is in, as well as the conductor's geometry and electric potential (i.e., voltage) in comparison to the surrounding medium. Many liquid mediums have a non-negligible concentration of unbound (i.e., mobile) charged particles in the form of ions. By way of example, when salt is dissolved in water, it splits into sodium and chloride charged particles Na+ and Cl−. The conductivity of the water is proportional to the concentration of ions in the water—increasing the concentration of ions increases the water conductivity.

According to example implementations of the disclosed technology, the total ion current collected by an electrically conducting object can be measured in detail at a range of conductor voltages. The resulting I-V relationship can be determined and used to determine physical property details such as electric potential, ion concentration, ion composition, and liquid temperature. Consider an example where the conductor is first biased to the potential of the liquid in which it is located. In this situation, very little ion current will be collected by the conductor as the ions are neither attracted to nor repelled from the surface of the conductor. The positive and negative fluxes will substantially cancel each other out and little to no ion current will be collected by the conductor. Consider now that the conductor is biased to a high positive voltage. In this situation, positive ions are repelled from and negative ions are attracted to the surface of the conductor. As a result, a large ion current will be collected by the conductor. The sensor system can determine an I-V characteristic such as an I-V curve based on the ion currents and voltages. The sensor system can analyze the I-V curve to determine a local potential of the liquid, such as by analyzing the slope of the I-V curve. In particular, a local peak or minimum of a derivative of the I-V curve can be used to determine a precise local potential.

The collected ion current to the conductor is also proportional to the liquid ion density of the liquid medium it is in. Accordingly, the control circuitry can be configured to determine the ion concentration of a liquid medium based on the I-V curve of the conductor. Additionally, the weight of ions can affect the collected ion current at the electrode and can thus be used to determine ion concentration and/or ion composition of the liquid medium. Heavier ions have a lower flux and are more difficult to attract to the conducting electrode surface which will reduce overall current. Accordingly, the control circuitry can be configured to determine the ion concentration and/or ion composition of a liquid medium based on the I-V curve of the conductor.

Further, the temperature of the liquid medium can affect the collected ion current at the electrode and can thus be used to determine the liquid temperature. At higher temperatures, ions will have higher average velocities such that the current collected by the electrode is more randomized and spread out across the voltage range applied to the electrode. Accordingly, the control circuitry in example implementations can be configured to determine liquid temperature based on the I-V curve.

Embodiments in accordance with the present disclosure provide a number of technical effects and benefits, particularly in the area of instrument and sensing technology. A sensor system for measuring the physical properties of liquids is described that provides multiple property measurements in a simple and robust configuration. Traditional sensor systems for measuring liquid properties often include electrodes or probes that are capable of measuring a single physical property of a test liquid. To test additional properties, additional systems or electrodes are required. In addition, these systems often require regular maintenance. For instance, some systems rely on chemical reactions which necessitate calibration and/or replenishing test materials as compounds are depleted. When multiple electrodes are included, the instruments are not co-located which can lead to the inability to accurately measure spatial discrepancies. Moreover, these systems can be fragile and complex.

In accordance with example embodiments of the present disclosure, a sensor system includes a sensor electrode formed from a simple electrically conductive object and control circuitry that is configured to leverage the sensor electrode for multiple physical property measurements. The use of an electrically conducting object and select bias conditions enables the determination of an I-V characteristic from which multiple physical property determinations can be made using a single electrode. Supporting circuitry can determine liquid properties using computationally-inexpensive algorithms based on modeled ion flows. In such a fashion, the system can accurately measure liquid electric potential, ion concentration, ion composition, and liquid temperature. The conductor is simple and mechanically robust, for example, being formed of a corrosion-resistant material having a simple geometry. The system does not require the test liquid to be brought into the measurement device, increasing longevity and decreasing complexity. This improved and simplified sensor system can improve measurements in applications such as science, human safety, environmental, healthcare, chemistry, manufacturing and others.

With reference now to the Figures, example embodiments of the present disclosure will be discussed in further detail.

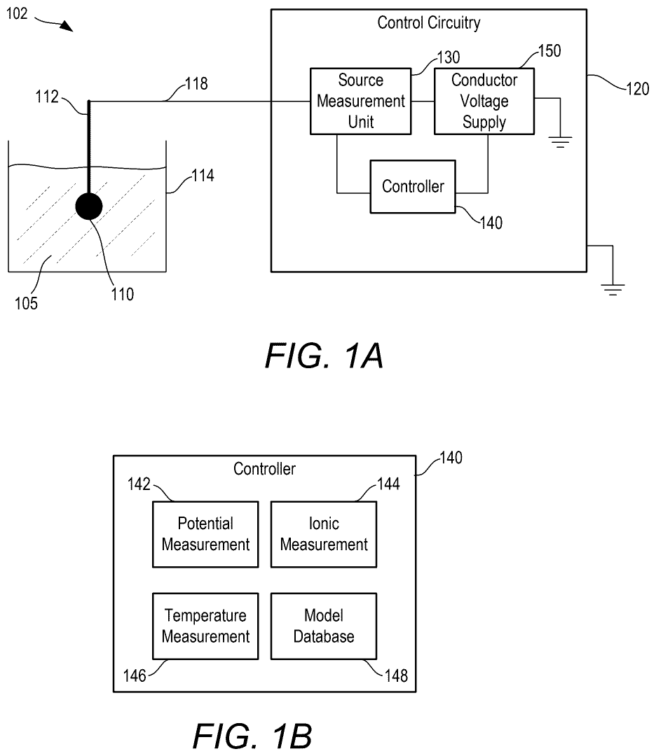

FIG. 1A is a block diagram depicting an example sensor system 102 configured to measure one or more physical properties of test liquids in accordance with example embodiments of the present disclosure. Sensor system 102 includes a measurement electrode comprising a conductor 110 that is configured for placement in contact with a test liquid 105. Conductor 110 is in electrical communication with control circuitry 120 which is configured to bias the conductor, determine a collected ion current, and determine one or more physical properties of the test liquid 105 based at least in part on the conductor biasing and collected ion current.

The measurement electrode includes a conductor 110 which can be a conducting object having a defined geometry. By way of example, conductor 110 can include a spherical geometry, cylindrical geometry, planar geometry, or other defined geometry. Conductor 110 may be made of a conducting material such as a metal (e.g., steel, copper, silver) or graphite, etc. Conductor 110 can be placed into or otherwise located within a container 114 containing a test liquid 105 for which one or more physical properties are to be determined. Container 114 can be any suitable container for holding a liquid. Conductor 110 is submerged within liquid 105 in the example of FIG. 1. Container 114 may be formed of a metal such as steel or copper or another material. It is noted that a container is not required. For example, conductor 110 can be arranged such that it can come into contact with a body of water, such as a pool, lake, ocean, pond, etc.

Conductor 110 is electrically coupled to control circuitry 120 of sensor system 100 through one or more conductors 118. For example, measurement conductor 110 can be electrically connected to control circuitry 120 via a conductive wire or other conducting element. The conducting element can be covered by an insulating sheath 112 at one or more portions that may come in contact with liquid 105 and/or container 114. Sheath 112 can avoid electrical shorting the conductor 118 to container 114 or liquid 105. Additionally, sheath 112 can prevent ion currents to the supporting rod that is submerged in the liquid. Currents to an exposed support structure may also be measured if not electrically insulated, reducing measurement accuracy and distorting the I-V curve.

Control circuitry 120 includes a source measurement unit 130 and conductor voltage supply 150 electrically coupled to conductor 110. Source measurement unit 130 can include one or more circuits configured to determine a collected ion current associated with conductor 110. For example, source measurement unit 130 can include a sourcemeter, ammeter, or other circuit(s) configured to determine a current amperage of the measurement conductor 110 under the applied bias conditions.

Conductor voltage supply 150 is electrically coupled to conductor 110 via a series connection with source measurement circuit. Other circuit architectures can be used in example embodiments.

Control circuitry 120 includes a controller 140 configured to communicate with source measurement unit 130 and conductor voltage supply 150 to bias and determine characteristics of the measurement conductor 110. Controller 140 is configured to control voltage supply 150 to bias conductor 110 to at least two voltages. Voltage supply 150 can apply a voltage sweep including multiple voltages to the measurement conductor 110.

In example embodiments, voltage supply 150 can apply a voltage sweep of progressively increasing voltages. The voltage sweep can include at least one positive voltage and at least one negative voltage with respect to the liquid potential or the control circuit in example implementations. In another example, the voltage supply 150 can apply a voltage sweep of progressively decreasing voltages. In an example implementation, the voltage supply 150 can apply the voltage sweep as a direct current (DC) signal including a plurality of discrete direct current (DC) voltage steps. For example, the voltage supply can apply a series of progressively increasing voltage steps between −10V and 10V as discrete voltage steps in increments of 1V. In another example implementation, the voltage supply 150 can apply the voltage sweep as an alternating current (AC) signal that oscillates between a negative peak voltage (e.g., −10V) and a positive peak voltage (e.g., +10V).

Source measurement unit 130 is configured to determine precise measurements of the total collected ion current by conductor 110 at each of the applied voltages. If discrete DC voltage steps are applied by voltage supply 150, source measurement unit 130 can measure the resulting ion current to conductor 110 at each voltage step. If a continuous AC voltage is applied by voltage supply 150, source measurement unit 130 can measure the resulting ion current to conductor 110 as the voltage oscillates between the negative peak voltage and the positive peak voltage.

Controller 140 can be configured to obtain the measurements of the total collected ion current from source measurement unit 130 and determine one or more physical properties of the test liquid 105. Controller 140 can determine and optionally record the ion current of conductor 110 measured by source measurement unit 130 at each voltage applied by voltage supply 150. Controller 140 can determine a current-voltage (I-V) curve and/or one or more other I-V characteristics associated with the test liquid based on the conductor biasing. Controller 140 can analyze the I-V curve to determine important properties of the test liquid 105 including the local voltage proximate to the conductor 110, the ionic concentration (e.g., salinity), ionic composition, and/or temperature with sensitive measurements.

FIG. 1B is a block diagram depicting an example implementation of control circuitry for a sensor system configured to measure one or more physical properties of liquids according to example embodiments of the present disclosure. FIG. 1B depicts an example of controller 140 including a potential measurement component 142, ionic measurement component 144, temperature measurement component 146, and model database 148. Controller 140 is one example implementation of controller 140 depicted in FIG. 1. Controller 140 can be implemented in hardware, software, or combinations of hardware and software. By way of example, controller 140 can be implemented by one or more processors and/or one or more dedicated circuits. Controller 140 may be implemented in general or dedicated hardware such as application-specific integrated circuits, programmable-gate arrays, and other hardware configurations.

Potential measurement component 142 is configured to determine a local potential of a test liquid proximate to a measurement electrode including a conductor such as conductor 110. In an example implementation, potential measurement component 142 can access or otherwise determine an I-V characteristic such as an I-V curve of the test liquid based on the relationship of measured ion current to the conductor voltage from the applied bias conditions. In an example implementation, the potential measurement component 142 can access one or more models or other data indicative of properties of the type of test liquid, the properties of the conductor (e.g., geometry, material), etc. The models or other data associated with the measurement system can be stored in a model database 148 or any memory or storage accessible by the controller. The potential measurement component 142 can determine a local potential of the test liquid based on the slope of the I-V curve. For example, the potential measurement component can analyze the slope of the I-V curve to determine the local potential of the test liquid. The potential measurement component can determine a derivative of the I-V curve and examine the resulting derivative for local peaks/minimums. For example, the controller can select the first minimum that is higher than the probe floating potential and determine that the probe voltage corresponding to the first minimum is the local potential of the liquid.

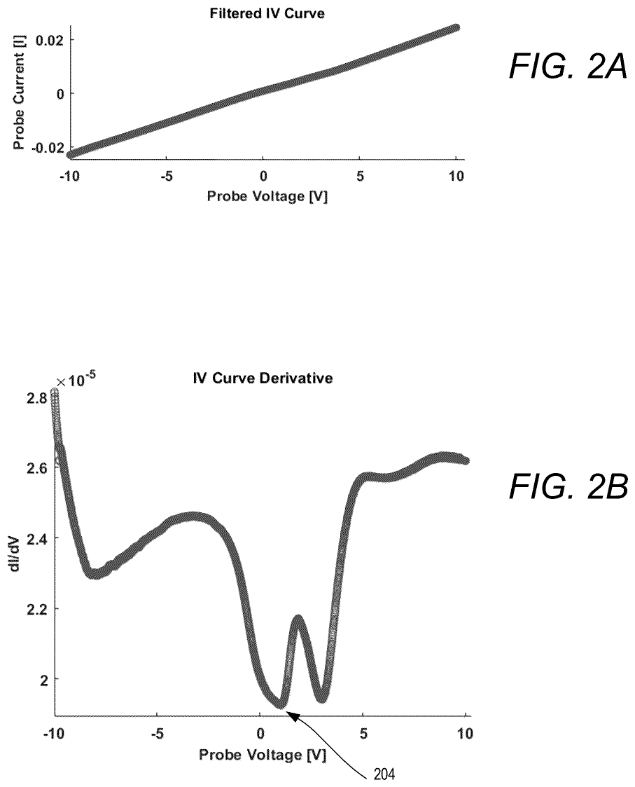

FIG. 2A is a diagram depicting an example current-voltage curve of a test liquid as measured by a sensor system for measuring the physical properties of liquids according to an example embodiment of the present disclosure. FIG. 2A depicts an I-V curve having a relatively constant slope as the probe voltage is swept from a negative voltage of −10V to a positive voltage of +10V. Notably, however, a slight kink is present in the I-V curve in the range of about 0V to 5V. This kink is more visible in the derivative of the I-V curve shown in FIG. 2B. The additional level of analysis described herein enables measuring the local potential, temperature, etc. accurately using just a single electrode. The resulting probe current representing the collected ion current increases from −0.02 A to +0.02 A as the voltage is swept from −10V to 10V.

FIG. 2B depicts an I-V curve derivative that can be generated by the potential measurement circuitry by taking the derivative of the I-V curve in FIG. 2A. The I-V curve derivative includes multiple local peaks/minimums. The potential measurement component 142 can determine the local potential of the liquid based on one or more of the local peaks/minimums of the derivative. For example, the system can select the first minimum that is higher than the probe floating potential and determine that the probe voltage corresponding to the first minimum is the local potential of the liquid. The first minimum higher than the probe floating potential is shown at 204 which corresponds to a probe voltage of approximately 1.0V. Accordingly, the potential measurement component 142 can determine that the local potential of the liquid is 1.0V in this example.

Ionic measurement component 144 is configured to determine the ionic concentration and/or ionic composition of a test liquid proximate to the measurement conductor. Ionic measurement component 144 can access or otherwise determine an I-V characteristic such as an I-V curve of the test liquid based on the relationship of measured ion current to the conductor voltage from the applied bias conditions. The ionic measurement component 144 can access one or more models or other data indicative of properties of the type of test liquid, the properties of the conductor (e.g., geometry, material), etc. stored in model database 208. The total ion current can be defined by the conductor geometry as well as the properties of the test liquid. The collected ion current is proportional to the liquid ion density. Heavier ions have a lower flux and are more difficult to attract to the conductor surface which reduces overall current.

Based on the proportionality of ion current to liquid ion density, ionic measurement component 144 can utilize the I-V curve to determine ionic concentrations and/or ionic composition of test liquids. The generated I-V curve can be compared to the modeled characteristics of potential concentrations and compositions to fit the I-V curve to a model and thereby determine an ionic concentration and/or ionic composition.

Temperature measurement component 146 includes one or more electrical circuits configured to determine the temperature of a test liquid proximate to the measurement conductor. Temperature measurement component 146 can access or otherwise determine an I-V characteristic such as an I-V curve of the test liquid based on the relationship of measured ion current to the conductor voltage from the applied bias conditions. The ionic measurement component 144 can access one or more models or other data indicative of properties of the type of test liquid, the properties of the conductor (e.g., geometry, material), etc. stored in model database 148. The total ion current can be defined by the conductor geometry as well as the properties of the test liquid. As earlier noted, ion current is proportional to the liquid ion density. Like ion density and weight, liquid temperature can affect the total ion current. By way of example, higher temperature liquids result in ions with higher average velocities which results in a more randomized and larger spread of collected current across the bias voltage.

Based on the proportionality of ion current to liquid temperature, temperature measurement component 146 can utilize the I-V curve to determine the temperature of test liquids. The generated I-V curve can be compared to the modeled characteristics of liquid temperatures to fit the I-V curve to a model and thereby determine a temperature of the test liquid.

The example sensor system 100 depicted in FIGS. 1A and 1B is one example of a measurement system in accordance with example embodiments of the present disclosure. It is noted that other example implementations are possible in accordance with embodiments of the present disclosure. By way of example, a measurement system in some examples may not include a container 114 but may be configured such that measurement conductor 110 directly contacts liquids without having a separate container. For instance, a measurement system can be configured with a conductor 110 physically coupled to an object (e.g., a submersible vehicle) such that measurements can be taken as the object moves or is otherwise placed within a liquid medium.

It is noted that the slope of the I-V curve in FIG. 2A is relatively constant at all probe voltages. There is a slight change in slope at a transition region between about 1V and 5V. The constant slope of the I-V curve is representative of the ion composition and ion concentration of the test liquid. For example, consider that the test liquid is water having dissolved salt therein. The salt splits into charged Na+ and Cl− particles as the salt is dissolved in the water. When the conductor 110 is biased to a negative voltage, the conductor will attract positively charged Na+ particles and repel negatively charged Cl− particles in proportion to the level of the negative voltage. When the conductor 110 is biased to a positive voltage, the conductor will attract negatively charged Cl− particles and repel positively charged Na+ particles in proportion of the level of the positive voltage. Accordingly, the resulting I-V curve has a relatively constant increase in probe current from a negative level to a positive level as the conductor voltage is swept from a negative voltage to a positive voltage.

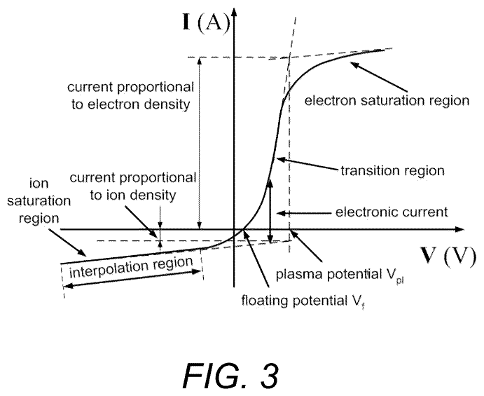

The I-V curve of a conductor under a sweeping voltage bias is similar for liquids such as water and plasmas. FIG. 3 is an example I-V curve of a test plasma when subjected to similar biasing as the liquid example of FIG. 2A. For a plasma, the I-V curve demonstrates different properties or regions based on the ion and electron saturations. At large negative voltages, the I-V curve for plasma demonstrates an ion saturation region for the plasma where the current is proportional to ion density in this interpolation region. A transition region exists between a small negative voltage and a small positive voltage. At larger positive voltages, the plasma enters an electron saturation region in which the current is proportion to electron density.

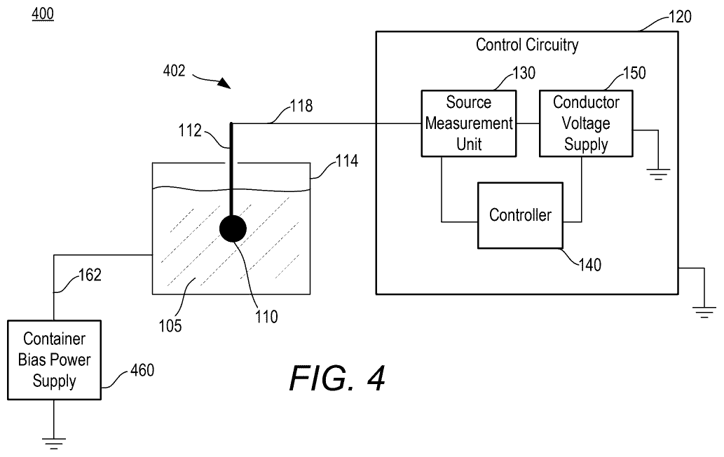

FIG. 4 is a block diagram of a test environment 400 including a liquid property measurement system in accordance with example embodiments of the present disclosure. Test environment includes a sensor system 402 including a conductor 110 electrically coupled to control circuitry 120. Sensor system 402 is one example of sensor system 102 depicted in FIG. 1 and is operative in the same manner as system 102.

Test environment 400 additionally includes a container bias power supply 460 electrically coupled to a container 114 via one or more electrical connections 162 (e.g., wire). Container 114 can be formed of a conducting material such as a metal (e.g., steel, copper, silver) or other conductive material (e.g., graphite). Container bias power supply 460 can apply voltages to the container. As shown in FIG. 4, container bias power supply 460 is electrically coupled to container 114. Power supply 460 can apply voltages to container 114 in order to bias the container 114 and test liquid 105 to a desired potential. For example, container 114 can be made to electrically float by insulating the container from any adjacent surfaces. While it is electrically floating, biasing container 114 will bias adjacent particles including liquid 105 to the same potential as container 114.

Accordingly, container bias power supply 460 can apply a voltage to container 114 to cause the test liquid 105 to be biased to a particular voltage. In example embodiments, power supply 460 can apply a voltage sweep of progressively increasing voltages. The voltage sweep can include at least one positive voltage and at least one negative voltage in example implementations. For example, the voltage supply can apply a series of voltages between −50V and 50V as discrete voltage steps in increments of 1V.

With the electrically floating container 114 biased to a particular voltage, control circuitry 120 can bias the conductor 110 and measure the collected ion current as previously described. For example, control circuitry 120 can apply a voltage sweep that sweeps through negative and positive voltages and determine the collected ion current. Control circuitry 120 can analyze the resulting I-V curve and determine a local potential of the test liquid 105.

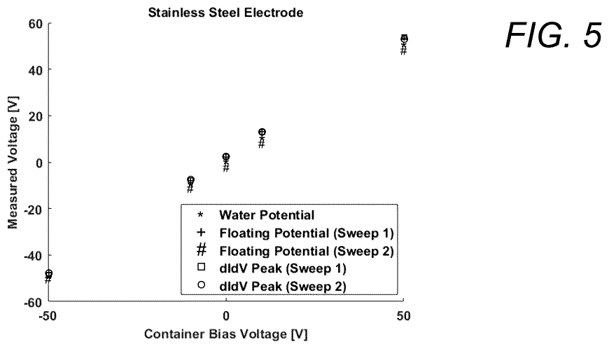

FIG. 5A is a diagram including a graphical depiction of example test results from a test applying a known potential to a test liquid and measuring the liquid potential using a sensor system in accordance with example embodiments of the present disclosure. FIG. 5A depicts a graph with a container bias voltage (V) plotted along the x-axis and a measured voltage (V) plotted along the y-axis. The container bias voltage is swept between −50V and +50V. The container is electrically floating so that the test liquid is biased to the same voltage as the container. With the test liquid biased to a particular voltage, the liquid measurement system determines the liquid potential using a conductor and the biasing/measurement techniques as described. FIG. 5A depicts the results from two separate sweeps by the sensor system. Specifically, FIG. 5A depicts the measured liquid potential (e.g., water potential), the floating potential of the container during each sweep, and the dl/dV peak for each sweep. As FIG. 5A illustrates, the voltage of the test liquid is accurately measured by the sensor system across the range of applied container bias voltages. FIG. 5A depicts the results of a test experiment conducted with a stainless steel conductor electrode. Similar results occur with different types of conductor electrodes.

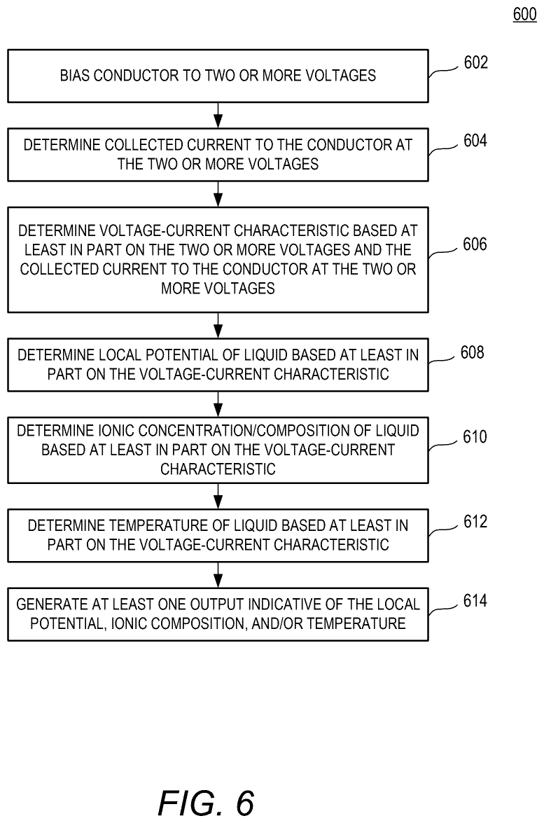

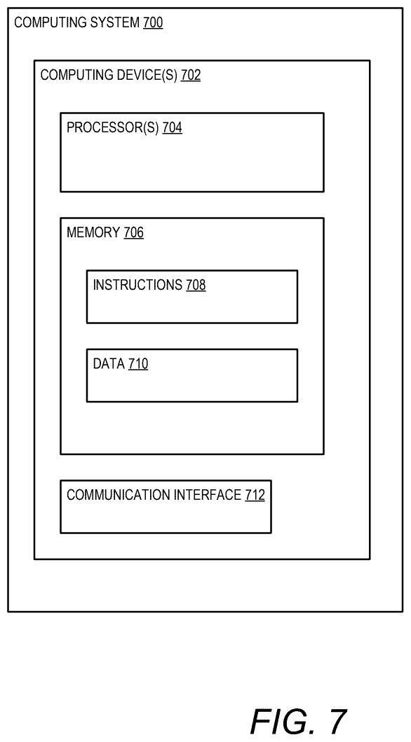

FIG. 6 is a flowchart depicting an example method 600 of measuring one or more physical properties of a liquid according to an example embodiment of the present disclosure. One or more portions of method 600 can be implemented by a sensor system including control circuitry and a measurement electrode such, such as, for example, control circuitry 120 and a measurement electrode including conductor 110 as depicted in FIG. 1. One or more portions of method 600 described herein can be implemented as an algorithm on the hardware components of the devices described herein (e.g., in circuitry as described in FIG. 1 or a computing system as described in FIG. 7) to, for example, measure one or more physical properties of a liquid such as water. Although FIG. 6 depicts steps performed in a particular order for purposes of illustration and discussion, method 600 and the other methods described herein are not limited to the particularly illustrated order or arrangement. The various steps of the methods disclosed herein can be omitted, rearranged, combined, and/or adapted in various ways without deviating from the scope of the present disclosure.

At 602, method 600 can include biasing a measurement conductor to two or more voltages. In example implementations, method 600 can include biasing the measurement conductor to three or more, or many more voltages. Biasing the conductor to two or more voltages may result in a linear assumption, for example when determining salinity. Accordingly, additional bias voltages can be used to exploit how the I-V curve deviates from linear. For example, the system can bias the measurement conductor to three or more voltages to determine a non-linearity of the I-V curve. The measurement conductor can have a defined geometry (e.g., sphere, cylinder, plane, etc.). The measurement conductor can be biased by applying a voltage sweep including multiple positive and/or negative voltages to the measurement conductor 110. The voltage sweep can include progressively increasing voltages that include at least one positive voltage and at least one negative voltage in example implementations. The voltage sweep can be applied as a plurality of discrete direct current (DC) voltage steps or as an AC voltage that oscillates between a negative peak voltage and a positive peak voltage.

At 604, method 600 can include determining the ion current to the measurement conductor at the voltages applied at 602. While the measured current may include the total current, practically the total current is dominated by ions for most liquids. Precise measurements of the total ion current collected by the conductor can be determined at each of the applied voltages. If discrete DC voltage steps are applied, the resulting ion current to the measurement conductor can be measured at each voltage step. If a continuous AC voltage is applied, the resulting ion current to the measurement conductor can be measured as the voltage oscillates between the negative peak voltage and the positive peak voltage.

At 606, method 600 can include determining a voltage-current (I-V) characteristic based at least in part on the voltages applied to the measurement conductor and the collected current to the conductor at the applied voltages. The total ion current can be defined by the conductor geometry as well as the properties of the test liquid. Based on the total collected ion current, a current-voltage (I-V) curve and/or one or more other I-V characteristics associated with the test liquid can be determined based on the conductor biasing.

At 608, method 600 can include determining a local potential of the test liquid based at least in the part on the voltage-current characteristic. One or more models or other data indicative of known liquid characteristics, conductor geometry, material, etc. can be used to determine the local potential in example embodiments. A local potential of the test liquid can be determined based on the slope of the I-V curve during biasing. For example, the slope of the I-V curve can be analyzed to determine the local potential of the test liquid, such as by determining a derivative of the I-V curve, identifying one or more local peaks/minimums, and selecting one or more peaks/minimums. For example, the system can select the first minimum that is higher than the probe floating potential and determine that the probe voltage corresponding to the first minimum is the local potential of the liquid.

At 610, method 600 can include determining an ionic concentration and/or ionic composition of the test liquid based at least in the part on the voltage-current characteristic. Similar to the local potential determination, one or more models or other data indicative of known liquid characteristics, conductor geometry, material, etc. can be used to determine the ionic concentrations and/or ionic compositions. The ionic concentration and/or ionic composition of the test liquid can be determined based on the slope of the I-V curve during biasing. For example, the slope of the I-V curve can be analyzed to determine an ionic concentration and/or ionic composition. The total ion current can be defined by the conductor geometry as well as the properties of the test liquid and the collected ion current can be proportional to the liquid ion density. Heavier ions have a lower flux and are more difficult to attract to the conductor surface which can reduce overall current. Based on the proportionality of ion current to liquid ion density, the ionic concentration and/or ionic composition of the test liquid can be determined.

At 612, method 600 can include a temperature of the test liquid based at least in the part on the voltage-current characteristic. Again, one or more models or other data indicative of known liquid characteristics, conductor geometry, material, etc. can be used to determine the temperature of the test liquid. The temperature of the test liquid can be determined based on the slope of the I-V curve during biasing. Heavier ions have a lower flux and are more difficult to attract to the conductor surface which can reduce overall current. Based on the proportionality of ion current to liquid ion density, the ionic concentration and/or ionic composition of the test liquid can be determined. Liquid temperature can affect the total ion current such that higher temperature liquids can result in ions with higher average velocities. Higher velocity ions in turn result in a more randomized and larger spread of collected current across the bias voltage. Based on the proportionality of ion current to liquid temperature, the temperature of the test liquid can be determined.

At 614, method 600 can include generating at least one output indicative of the local potential, the ionic composition, the ionic concentration, and/or the temperature of the test liquid. It is noted that while method 600 describes a determination of local potential, ionic composition, ionic concentration, and/or temperature of the test liquid, this is not required. For example, a sensor system can be configured to determine one or more of the local potential, ionic composition, ionic concentration, and/or temperature of the test liquid. It is noted that steps 608, 610, and 612 do not need to be performed in every instance. These steps can be performed sequentially, in parallel, various orders, and according to the needs and priorities of a particular implementation.

FIG. 7 depicts a block diagram of an example computing system 700 that can be used by a tracking control system, mobile computing device, or other systems to implement methods and systems according to example embodiments of the present disclosure. As shown, the computing system 700 can include one or more computing device(s) 702. The one or more computing device(s) 702 can include one or more processor(s) 704 and one or more memory device(s) 706. The one or more processor(s) 704 can include any suitable processing device, such as a microprocessor, microcontroller, integrated circuit, logic device, or other suitable processing device. The one or more memory device(s) 706 can include one or more computer-readable media, including, but not limited to, non-transitory computer-readable media, RAM, ROM, hard drives, flash drives, or other memory devices.

The one or more memory device(s) 706 can store information accessible by the one or more processor(s) 704, including computer-readable instructions 708 that can be executed by the one or more processor(s) 704. The memory device(s) can store instructions 708 which can be any set of instructions that when executed by the one or more processor(s) 704, cause the one or more processor(s) 704 to perform operations. The instructions 708 can be software written in any suitable programming language or can be implemented in hardware. In some embodiments, the instructions 708 can be executed by the one or more processor(s) 704 to cause the one or more processor(s) 704 to perform operations, such as the operations for biasing a measurement electrode, measuring collected ion current by a conductor of the electrode, and determining one or more physical properties of a liquid as described above, and/or any other operations or functions of the one or more computing device(s) 702.

The memory device(s) 706 can further store data 710 that can be accessed by the processors 704. For example, the data 710 can include state data, association data, processing cycle and/or stages data, and user interface data, etc., as described herein. The data 710 can include one or more table(s), function(s), algorithm(s), model(s), equation(s), etc. according to example embodiments of the present disclosure.

The one or more computing device(s) 702 can also include a communication interface 712 used to communicate, for example, with the other components of system. The communication interface 712 can include any suitable components for interfacing with one or more network(s), including for example, transmitters, receivers, ports, controllers, antennas, or other suitable components.

The technology discussed herein makes reference to computer-based systems and actions taken by and information sent to and from computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Embodiments may be implemented by hardware, software, firmware, middleware, microcode, hardware description languages, and/or any combination thereof. When implemented in software, firmware, middleware, scripting language, and/or microcode, the program code or code segments to perform the necessary tasks may be stored in a machine-readable medium such as a storage medium. Embodiments may be implemented in general or dedicated hardware such as application-specific integrated circuits, programmable-gate arrays, and other hardware configurations.

Although specific features of various embodiments may be shown in some drawings and not in others, this is for convenience only. In accordance with the principles of the present disclosure, any feature of a drawing may be referenced and/or claimed in combination with any feature of any other drawing.

This written description uses examples to disclose the claimed subject matter, including the best mode, and also to enable any person skilled in the art to practice the claimed subject matter, including making and using any devices or systems and performing any incorporated methods. The patentable scope of the disclosed technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method of measuring physical properties of liquids, comprising:
providing a conductor of a defined geometry in contact with a liquid;
biasing the conductor to two or more voltages;
determining a collected current to the conductor at the two or more voltages;
determining at least one current-voltage characteristic based at least in part on the two or more voltages and the collected current to the conductor at the two or more voltages;
determining at least one physical property of the liquid proximate to the conductor based at least in part on the at least one current-voltage characteristic; and
generating at least one output indicative of the at least one physical property of the liquid proximate to the conductor.

2. The method of claim 1, wherein:
determining at least one physical property of the liquid proximate to the conductor comprises determining a potential of the liquid proximate to the conductor based at least in part on the at least one current-voltage characteristic; and
generating at least one output indicative of the at least one physical property of the liquid proximate to the conductor comprises generating at least one output indicative of the potential of the liquid proximate to the conductor.

3. The method of claim 1, further comprising:
determining at least one physical property of the liquid proximate to the conductor comprises determining a temperature of the liquid proximate to the conductor based at least in part on the at least one current-voltage characteristic; and
generating at least one output indicative of the at least one physical property of the liquid proximate to the conductor comprises generating at least one output indicative of the temperature of the liquid proximate to the conductor.

4. The method of claim 1, further comprising:
determining at least one physical property of the liquid proximate to the conductor comprises determining at least one of an ionic concentration of the liquid proximate to the conductor or an ionic composition based at least in part on the at least one current-voltage characteristic; and
generating at least one output indicative of the at least one physical property of the liquid proximate to the conductor comprises generating at least one output indicative of at least one of the ionic concentration or the ionic composition the liquid proximate to the conductor.

5. The method of claim 4, wherein:
the liquid is water;
the water includes sodium and chloride; and
the at least one of the ionic concentration or the ionic composition is based on a concentration of the sodium and the chloride.

6. The method of claim 1, wherein biasing the conductor to two or more voltages comprises:
providing a voltage sweep to the conductor including progressively increasing voltage steps, the progressively increasing voltage steps including at least one negative voltage and at least one positive voltage relative to a local potential of the liquid.

7. The method of claim 1, wherein biasing the conductor to two or more voltages comprises:
providing a voltage sweep to the conductor including progressively decreasing voltage steps, the progressively decreasing voltage steps including at least one negative voltage and at least one positive voltage relative to a local potential of the liquid.

8. The method of claim 1, wherein biasing the conductor to two or more voltages comprises:
biasing the conductor to three or more voltages to determine a non-linearity associated with the at least one current-voltage characteristic.

9. The method of claim 1, wherein biasing the conductor to two or more voltages comprises:
biasing the conductor with an alternating current signal, the two or more voltages including negative and positive voltages resulting from the alternating current signal.

10. The method of claim 1, wherein the conductor includes a metal.

11. The method of claim 1, wherein the defined geometry of the conductor is at least one of a sphere, a cylinder, or a plane.

12. The method of claim 1, further comprising:
collecting charged particles at a surface of the conductor based at least in part on biasing the conductor to two or more voltages.

13. A sensor system to measure physical properties of liquids, comprising:
a conductor having a defined geometry; and
control circuitry in electrical communication with the conductor, the control circuitry being configured to bias the conductor to two or more voltages, determine a collected current to the conductor at the two or more voltages, determine at least one current-voltage characteristic based at least in part on the two or more voltages and the collected current to the conductor at the two or more voltages, determine at least one physical property of the liquid proximate to the conductor based at least in part on the at least one current-voltage characteristic, and generate at least one output indicative of the at least one physical property of the liquid proximate to the conductor.

14. The sensor system of claim 13, wherein:
the control circuitry is configured to determine at least one physical property of the liquid proximate to the conductor by determining a potential of the liquid proximate to the conductor based at least in part on the at least one current-voltage characteristic; and
the control circuitry is configured to generate at least one output indicative of the at least one physical property of the liquid proximate to the conductor by generating at least one output indicative of the potential of the liquid proximate to the conductor.

15. The sensor system of claim 13, wherein:
the control circuitry is configured to determine at least one physical property of the liquid proximate to the conductor by determining a temperature of the liquid proximate to the conductor based at least in part on the at least one current-voltage characteristic; and
the control circuitry is configured to generate at least one output indicative of the at least one physical property of the liquid proximate to the conductor by generating at least one output indicative of the temperature of the liquid proximate to the conductor.

16. The sensor system of claim 13, wherein:
the control circuitry is configured to determine at least one physical property of the liquid proximate to the conductor by determining an ionic concentration of the liquid proximate to the conductor based at least in part on the at least one current-voltage characteristic; and
the control circuitry is configured to generate at least one output indicative of the at least one physical property of the liquid proximate to the conductor by generating at least one output indicative of the ionic concentration of the liquid proximate to the conductor.

17. The sensor system of claim 13, wherein the control circuitry is configured to bias the conductor to two or more voltages by providing a voltage sweep to the conductor including progressively increasing voltage steps, the progressively increasing voltage steps including at least one negative voltage and at least one positive voltage.

18. The sensor system of claim 13, wherein the control circuitry is configured to bias the conductor to two or more voltages by biasing the conductor with an alternating current signal, the two or more voltages including negative and positive voltages resulting from the alternating current signal.

19. The sensor system of claim 13, wherein the defined geometry of the conductor is at least one of a sphere, a cylinder, or a plane.

20. One or more non-transitory computer-readable media that store instructions that, when executed by one or more processors, cause the one or more processors to perform operations, the operations comprising:
biasing a conductor to two or more voltages while in contact with a liquid;
determining a collected ion current to the conductor at the two or more voltages;
determining at least one current-voltage characteristic based at least in part on the two or more voltages and the collected current to the conductor at the two or more voltages;
determining at least one physical property of the liquid proximate to the conductor based at least in part on the at least one current-voltage characteristic; and
generating at least one output indicative of the at least one physical property of the liquid proximate to the conductor.
